(19) United States
(12) Patent Application Publication
(10) Pub. No.: US 2013/0022564 A1
(43) Pub. Date: Jan. 24, 2013

(54) COMPOSITIONS AND METHODS FOR DERMALLY TREATING INFECTIONS

(71) Applicants: Jie Zhang, Salt Lake City, UT (US); Kevin S. Warner, West Jordan, UT (US); Sanjay Sharma, Sandy, UT (US)

(72) Inventors: Jie Zhang, Salt Lake City, UT (US); Kevin S. Warner, West Jordan, UT (US); Sanjay Sharma, Sandy, UT (US)

(21) Appl. No.: 13/556,089

(22) Filed: Jul. 23, 2012

Related U.S. Application Data
(63) Continuation of application No. 11/640,101, filed on Dec. 14, 2006, now abandoned.
Application No. 11/640,101 is a continuation-in-part of application No. 11/146,917, filed on Jun. 6, 2005.
(60) Provisional application No. 60/750,637, filed on Dec. 14, 2005.
(60) Provisional application No. 60/750,522, filed on Dec. 14, 2005.
(60) Provisional application No. 60/750,465, filed on Dec. 14, 2005.
(60) Provisional application No. 60/577,536, filed on Jun. 7, 2004.

(51) Int. Cl. A61K 31/522; A61P 31/12; A61P 31/10; A61K 31/4174

(52) U.S. Cl. 424/61; 514/263.38; 514/399

(57) ABSTRACT

The present invention is drawn to solidifying adhesive formulations, methods of drug delivery, and solidified layers for dermal delivery of a drug for treating various skin infections, such as fungal, bacterial, and/or viral skin infections. The formulation can include anti-infective drug, solvent vehicle, and solidifying agent. The solvent vehicle can include a volatile solvent system including at least one volatile solvent, and a non-volatile solvent system including at least one non-volatile solvent. The non-volatile solvent system can facilitate the delivery of the drug at therapeutically effective rates for sustained periods of time. The non-volatile solvent system can also act to plasticize the solidifying agent. The formulation can have a viscosity suitable for application to a skin surface prior to evaporation of the volatile solvents system. When applied to the skin, the formulation can form a solidified layer after at least a portion of the volatile solvent system is evaporated.

This application is a continuation of U.S. patent application Ser. No. 11/640,101, filed Dec. 14, 2006, and which claims the benefit of U.S. Provisional Application Nos. 60/750,637, 60/750,522, and 60/750,465, each of which was filed on Dec. 14, 2005, and is a continuation-in-part of U.S. application Ser. No. 11/146,917 filed on Jun. 6, 2005, which claims the benefit of U.S. Provisional Application No. 60/577,536 filed on Jun. 7, 2004, each of which is incorporated herein by reference.

### FIELD OF THE INVENTION

The present invention relates generally to systems and methods for treating various skin infections and/or pain associated therewith. More particularly, the present invention relates to solidifying adhesive formulations having a viscosity suitable for application to infected skin, and which form a sustained drug-delivering solidified adhesive layer on the skin.

### BACKGROUND OF THE INVENTION

Skin infections affect millions of people across the world. There are a variety of sources of infections including fungal, bacterial, and viral sources. For example, one type of viral infection is a herpes infection. Herpes infections often occur on lips, e.g. cold sores, and on the genitals. There are two common dosage forms available for treating cold sores and genital herpes, namely topical and oral. Both delivery forms have certain drawbacks. For example, oral delivery of acyclovir, an anti-viral drug, can cause undesirable side effects such as upset stomach, loss of appetite, nausea, vomiting, diarrhea, headache, dizziness, or weakness. One drawback of current topical anti-cold sore formulations, in the form of ointments and creams such as Zovirax ointment and cream, is that they are often inadvertently wiped off from the treatment site when the subject eats, drinks, or licks his/her lips, etc. This is believed to be a reason why topical cold sore formulations often need to be applied many times a day, which is very inconvenient and frequently results in poor patient compliance. When a topical anti-herpes formulation is applied on the genitals, the drug is often subject to inadvertent removal by underwear and adjacent healthy skin/mucosal surface contact. In addition, some topical formulations usually contain volatile solvent(s), such as water and ethanol, which tend to evaporate shortly after application. The complete evaporation of such solvents can cause a significant decrease or even termination of dermal drug delivery, thereby prematurely ending treatment. Additionally, semisolid formulations are often “rubbed into” the skin, which does not necessarily mean the drug formulation is actually delivered into the skin. Instead, this phrase often means that a very thin layer of the drug formulation is applied onto the surface of the skin. Such thin layers of traditional topical semisolid formulations may not contain sufficient quantity of active drug to achieve sustained delivery over long periods of time.

Fungal infections of the hair, nail, skin, and subcutaneous tissues are common diseases. They include nail fugal infections, athlete's foot, and certain kinds of diaper rashes. These diseases are currently treated with various kinds of topical medications. For example, nail fungus is treated with lacquers, such as Penlac™, which contains ciclopirox olamine, an antifungal agent; athlete's foot is treated with creams or solutions containing anti-fungal agents, such as Lamisil™. Diaper rashes are treated with ointments such as Desitin™. However, there are serious shortcomings with currently available treatment formulations. For example, after a layer of a lacquer is applied on the infected nail surface, all the solvent evaporates within a very short time, typically within one minute. After the complete or near complete evaporation of the solvent, the formulation layer becomes a rigid solid and the delivery rate of the drug is dramatically reduced or even stopped. After the intended treatment period, the subject typically has to wash the lacquer layer off with certain solvents, such as ethanol or acetone. A layer of cream applied to treat athlete's foot is subject to removal by objects such as socks. Similarly, a layer of Desitin™ cream applied on a baby's bottom is subject to removal by the diaper. Therefore, in general, current topical products for treating skin and nail fungal infections suffer from vulnerability of undesired removal from the treatment site, lack of sustained drug delivery, inconvenient removal after intended applications, and lack of protective physical barrier which can be beneficial in some cases.

Methods of treating bacterial infections, particularly bacterial infections of the skin, similarly suffer from the drawbacks of both the anti-fungal and anti-viral treatments.

In view of the shortcomings of current dermal anti-infective formulations, it would be desirable to provide systems, formulations, and/or methods that i) provide more sustained drug delivery over long periods of time, ii) are not vulnerable to unintentional removal by contact with clothing, iii) provide a protective physical barrier that is beneficial in certain disease treatment, and/or iv) are easily removed after application and use.

### SUMMARY OF THE INVENTION

It has been recognized that it would be advantageous to provide topical anti-infective formulations and related methods for the topical treatment of fungal, bacterial, and/or viral infections, which are capable of providing sustained release of drug and do not suffer from the drawback of unintentional removal. In accordance with this, the present invention is drawn generally to a formulation for treating an infection, comprising a drug that is effective for treating an infection, a solvent vehicle, and a solidifying agent. The solvent vehicle can comprise a volatile solvent system including at least one volatile solvent, and a non-volatile solvent system including at least one non-volatile solvent, wherein the non-volatile solvent system is capable of facilitating delivery of the drug at therapeutically effective rates over a sustained period of time. The formulation can have a viscosity suitable for application and adhesion to a skin surface prior to evaporation of the volatile solvent system. The formulation applied to the skin surface can form a soft, coherent, solidified layer after at least partial evaporation of the volatile solvent system. Further, the drug can continue to be delivered after the volatile solvent system is at least substantially evaporated.

In another embodiment, a method of treating an infection can comprise applying a solidifying adhesive formulation to an infected skin surface. The solidifying adhesive formulation can comprise a drug that is effective for treating an infection, a solvent vehicle, and a solidifying agent. The solvent system can comprise a volatile solvent system including at least one volatile solvent, and a non-volatile solvent system including at least one non-volatile solvent. The non-volatile solvent system can be capable of facilitating the delivery of the drug at therapeutically effective rates over a sustained period of time. The formulation can have a viscosity suitable for application and adhesion to the skin surface prior to evaporation of the volatile solvent system. Additional steps include solidifying the formulation to form a soft, coherent, solidified layer on the infected skin surface by at least partial evaporation of the volatile solvent system, and dermally delivering the drug from the solidified layer to the infected skin site at therapeutically effective rates over a sustained period of time.

In another embodiment, a soft, coherent, solidified layer for treating an infection can comprise a drug that is effective for treating an infection; a non-volatile solvent system including at least one non-volatile solvent, wherein the non-volatile solvent system facilitates the delivery of the drug at therapeutically effective rates over a sustained period of time; and a solidifying agent. The solidified layer can preferably be stretchable by 5% (or even 10%) in one direction without cracking, breaking, and/or separating from a skin surface to which the layer is applied.

In still another embodiment, a formulation for treating an infection can comprise a drug selected from the group consisting of acyclovir, valacyclovir, pencyclovir, and combinations thereof; a solvent vehicle comprising a volatile solvent system including at least one volatile solvent and a non-volatile solvent system comprising a non-volatile solvent; and a solidifying agent. The non-volatile solvent can be selected from the group consisting of oleic acid, isostearic acid, olive oil, and combinations thereof. The solidifying agent can be selected from the group consisting of ethyl acrylate-methyl methacrylate-trimethylammonioethyl methacrylate chloride copolymers, butyl and methyl methacrylate copolymers, ethyl cellulose, and mixtures and copolymers thereof. The formulation can have a viscosity suitable for application to a skin surface prior to evaporation of the volatile solvent system, can form a solidified, coherent, flexible, and continuous layer after at least partial evaporation of the volatile solvent system, and the drug can be continued to be delivered at a therapeutically effective rate after the volatile solvent system is at least substantially all evaporated.

In another embodiment, a formulation for treating an infection can comprise a drug selected from the group consisting of econazole, terbinafine, and combinations thereof; a solvent vehicle comprising a volatile solvent system including at least one volatile solvent and a non-volatile solvent system comprising at least one non-volatile solvent; and a solidifying agent. The non-volatile solvent can be selected from the group consisting of tetrahydroxypropyl ethylenediamine, oleic acid, isostearic acid, olive oil, and combinations thereof. The solidifying agent can be selected from the group consisting of ethyl acrylate-methyl methacrylate-trimethylammonioethyl methacrylate chloride copolymers, butyl and methyl methacrylate copolymers, ethyl cellulose, and mixtures and copolymers thereof. The formulation can have a viscosity suitable for application to a skin surface prior to evaporation of the volatile solvent system, can form a solidified, coherent, flexible, and continuous layer after at least partial evaporation of the volatile solvent system, and the drug can be continued to be delivered at a therapeutically effective rate after the volatile solvent system is at least substantially all evaporated.

In another embodiment, an adhesive solidifying formulation for treating a nail infection can comprise a drug that is effective for treating a nail infection, a solvent vehicle, and a solidifying agent. The solvent vehicle can comprise a volatile solvent system including at least one volatile solvent, and a non-volatile solvent system including at least one non-volatile solvent, wherein the non-volatile solvent system is capable of facilitating delivery of the drug at a therapeutically effective rate over a sustained period of time. The formulation has a viscosity suitable for application and adhesion to a nail surface prior to evaporation of the volatile solvent system, and when applied to the nail surface, it forms a solidified layer after at least partial evaporation of the volatile solvent system. Further, the drug continues to be delivered to the nail after the volatile solvent system is at least substantially evaporated.

In another embodiment, a method of treating nail fungal infection can comprise applying to a nail surface with a fungal infection, and optionally surrounding skin, a layer of an adhesive solidifying formulation. The formulation can comprise an anti-fungal drug, a solvent vehicle including a volatile solvent system comprising at least one volatile solvent, and a non-volatile solvent system comprising at least one non-volatile solvent, and a solidifying agent. The non-volatile solvent system can be capable of facilitating delivery of the anti-fungal drug at a therapeutically effective rate over a sustained period of time, and can have a viscosity suitable for application and adhesion to a nail surface prior to evaporation of the volatile solvent system. Further, the formulation applied to the nail surface can form a solidified layer after at least partial evaporation of the volatile solvent system, and the drug can continue to be delivered from the solidified layer to the nail after the volatile solvent system is at least substantially evaporated. Additional steps can include keeping the solidified layer on said nail surface for a treatment period of at least 4 hours, and removing the solidified layer after the treatment period.

Additional features and advantages of the invention will be apparent from the following detailed description and FIGURE which illustrate, by way of example, features of the invention.

### BRIEF DESCRIPTION OF THE DRAWING

FIG. 1 is a graphical representation of the cumulative amount of acyclovir delivered transdermally over time from two separate formulations in accordance with embodiments of the present invention compared to the marketed product Zovirax cream.

### DETAILED DESCRIPTION OF THE PREFERRED EMBODIMENT(S)

Before particular embodiments of the present invention are disclosed and described, it is to be understood that this invention is not limited to the particular process and materials disclosed herein as such may vary to some degree. It is also to be understood that the terminology used herein is used for the purpose of describing particular embodiments only and is not intended to be limiting, as the scope of the present invention will be defined only by the appended claims and equivalents thereof.

In describing and claiming the present invention, the following terminology will be used.

The singular forms “a,” “an,” and “the” include plural referents unless the context clearly dictates otherwise. Thus, for example, reference to “a drug” includes reference to one or more of such compositions.

“Skin” is defined to include human skin, nail, and mucosal surfaces that can suffer from bacterial, viral or fungal infections and are usually at least partially exposed to air such as standard skin, scalp, toe and finger nails, lips, genital and anal mucosa, and nasal and oral mucosa.

The term “drug(s)” refers to any bioactive agent or agents which can be used to effectively treat an infection, e.g., viral, fungal, and/or bacterial. This includes compositions that are traditionally identified as drugs, as well other bioactive agents that are not always considered to be “drugs” in the classic sense, but which can provide a therapeutic effect for certain conditions. In one embodiment, a single agent can be effective in treating multiple infection types. In another embodiment, multiple drugs for treating a single infection type can be concurrently present and delivered from the same solidified formulation. In another embodiment, multiple drugs targeting separate infection types can be delivered from the same solidified formulation.

Examples of antifungal drugs which can be used in the present invention include, but are not limited to, amorolfine, butenafine, naftifine, terbinafine, fluconazole, itraconazole, ketoconazole, posaconazole, ravuconazole, voriconazole, clotrimazole, butoconazole, econazole, miconazole, oxiconazole, sulconazole, terconazole, tioconazole, caspofungin, micafungin, anidulafingin, amphotericin B, AmB, nystatin, pimaricin, griseofulvin, ciclopirox olamine, haloprogin, tolnaftate, and undecylenate, or combinations thereof.

Examples of antiviral drugs which can be used in the present invention include, but are not limited to, acyclovir, penciclovir, famciclovir, valacyclovir, behenyl alcohol, trifluridine, idoxuridine, cidofovir, gancyclovir, podofilox, podophyllotoxin, ribavirin, abacavir, delavirdine, didanosine, efavirenz, lamivudine, nevirapine, stavudine, zalcitabine, zidovudine, amprenavir, indinavir, nelfinavir, ritonavir, saquinavir, amantadine, interferon, oseltamivir, ribavirin, rimantadine, zanamivir, or combinations thereof.

Examples of antibacterial drugs which can be used in the present invention include, but are not limited to, erythromycin, clindamycin, tetracycline, bacitracin, neomycin, mupirocin, polymyxin B, quinolones such as ciproflaxin, or combinations thereof. The active drug in the formulations and methods of the present invention for treating skin infections can also include immune modulating agents, including but is not limited to imiquimod.

The phrases “dermal drug delivery” or “dermal delivery of drug(s)” shall include both transdermal and topical drug delivery, and includes the delivery of drug(s) to, through, or into the skin. “Transdermal delivery” of drug can be targeted to skin tissues just under the skin, regional tissues or organs under the skin, systemic circulation, and/or the central nervous system. “Topical delivery” includes delivery of a drug to a skin tissue, and subsequent absorption into deeper tissues that may occur.

The term “flux” such as in the context of “dermal flux” or “transdermal flux,” respectively, refers to the quantity of the drug permeated into or across skin per unit area per unit time. A typical unit of flux is microgram per square centimeter per hour. One way to measure flux is to place the formulation on a known skin area of a human volunteer and measure how much drug can permeate into or across skin within certain time constraints. Various methods (in vivo methods) might be used for the measurements as well. The method described in Example 1 or other similar method (in vitro methods) can also be used to measure flux. Although an in vitro method uses human epidermal membrane obtained from a cadaver, or freshly separated skin tissue from hairless mice rather than measure drug flux across the skin using human volunteers, it is generally accepted by those skilled in the art that results from a properly designed and executed in vitro test can be used to estimate or predict the results of an in vivo test with reasonable reliability. Therefore, “flux” values referenced in this patent application can mean that measured by either in vivo or in vitro methods.

The term “flux-enabling” with respect to the non-volatile solvent system (or solidified layer including the same) refers to a non-volatile solvent system (including one or more non-volatile solvents) selected or formulated specifically to be able to provide therapeutically effective flux for a particular drug(s). For topically or regionally delivered drugs, a flux enabling non-volatile solvent system is defined as a non-volatile solvent system which, alone without the help of any other ingredients, is capable of delivering therapeutic effective levels of the drug across, onto or into the subject's skin when the non-volatile solvent system is saturated with the drug. For systemically targeted drugs, a flux enabling non-volatile solvent system is a non-volatile solvent system that can provide therapeutically effective daily doses over 24 hours when the non-volatile solvent system is saturated with the drug and is in full contact with the subject's skin with no more than 500 cm² contact area. In one embodiment, the contact area for the non-volatile solvent system is no more than 100 cm². Testing using this saturated drug-in-solvent state can be used to measure the maximum flux-generating ability of a non-volatile solvent system. To determine flux, the drug solvent mixture needs to be kept on the skin for a clinically sufficient amount of time. In reality, it may be difficult to keep a liquid solvent on the skin of a human volunteer for an extended period of time. Therefore, an alternative method to determine whether a solvent system is “flux-enabling” is to measure the in vitro drug permeation across the hairless mouse skin or human cadaver skin using the apparatus and method described in Example 1. This and similar methods are commonly used by those skilled in the art to evaluate permeability and feasibility of formulations. Alternatively, whether a non-volatile solvent system is flux-enabling can be tested on the skin of a live human subject with means to maintain the non-volatile solvent system with saturated drug on the skin, and such means may not be practical for a product. For example, the non-volatile solvent system with saturated drug can be soaked into an absorbent fabric material which is then applied on the skin and covered with a protective membrane. Such a system is not practical as a pharmaceutical product, but is appropriate for testing whether a non-volatile solvent system has the intrinsic ability to provide sufficient drug flux, or whether it is flux-enabling.

It is also noted that once the formulation forms a solidified layer, the solidified layer can also be “flux enabling” for the drug while some of the non-volatile solvents remain in the solidified layer, even after the volatile solvents (including water) have been substantially evaporated.

The phrase “effective amount,” “therapeutically effective amount,” “therapeutically effective rate(s),” or the like, as it relates to a drug, refers to sufficient amounts or delivery rates of a drug which achieves any appreciable level of therapeutic results in treating a condition for which the drug is being delivered. It is understood that “appreciable level of therapeutic results” may or may not meet any government agencies' efficacy standards for approving the commercialization of a product. It is understood that various biological factors may affect the ability of a substance to perform its intended task. Therefore, an “effective amount,” “therapeutically effective amount,” or “therapeutically effective rate(s)” may be dependent in some instances on such biological factors to some degree. However, for each drug, there is usually a consensus among those skilled in the art on the range of doses or fluxes that are sufficient in most subjects. Further, while the achievement of therapeutic effects may be measured by a physician or other qualified medical personnel using evaluations known in the art, it is recognized that individual variation and response to treatments may make the achievement of therapeutic effects a subjective decision. The determination of a therapeutically effective amount or delivery rate is well within the ordinary skill in the art of pharmaceutical sciences and medicine.

“Therapeutically effective flux” is defined as the permeation flux of the selected drug that delivers sufficient amount of drug into or across the skin to be clinically beneficial. It does not necessarily mean that most of the subject population can obtain some degree of benefit or the benefit is high enough to be deemed “effective” by relevant government agencies or the medical profession. More specifically, for drugs that target skin or regional tissues or organs close to the skin surface (such as joints, certain muscles, or tissues/organs that are at least partially within 5 cm of the skin surface), “therapeutically effective flux” refers to the drug flux that can deliver a sufficient amount of the drug into the target tissues within a clinically reasonable amount of time. For drugs that target the systemic circulation, “therapeutically effective flux” refers to drug flux that, via clinically reasonable skin contact area, can deliver sufficient amounts of the selected drug to generate clinically beneficial plasma or blood drug concentrations within a clinically reasonable time. Clinically reasonable skin contact area is defined as a size of skin application area that most subjects would accept. Typically, a skin contact area of 400 cm² or less is considered reasonable. Therefore, in order to deliver 4000 mcg of a drug to the systemic circulation via a 400 cm² skin contact area over 10 hours, the flux needs to be at least 4000 mcg/400 cm²/10 hour, which equals 1 mcg/cm²/hr. By this definition, different drugs have different “therapeutically effective flux.” Therapeutically effective flux” may be different in different subjects and or at different times for even the same subject. However, for each drug, there is usually a consensus among the skilled in the art on the range of doses or fluxes that are sufficient in most subjects at most times.

The following are estimates of flux for some drugs that are therapeutically effective or more than sufficient:

TABLE A
In vitro steady state flux values of various drugs

| | | Estimated |
|---|---|---|
| | | Therapeutically |
| | | effective flux* |
| Drug | Indication | (mcg/cm²/h) |
| Ropivacaine** | Neuropathic pain | 5 |
| Lidocaine | Neuropathic pain | 30 |
| Acyclovir | Herpes simplex virus | 3 |
| Ketoprofen | Musculoskeletal pain | 16 |
| Diclofenac | Musculoskeletal pain | 1 |
| Clobetasol | Dermatitis, psoriasis, | 0.05 |
| | eczema | |
| Betamethasone | Dermatitis, psoriasis, | 0.01 |
| | eczema | |
| Testosterone | Hypogonadal men, | 0.8 |
| Testosterone | Hormone treatment for | 0.25 |
| | postmenopausal | |
| | women | |
| Imiquimod | Warts, basal cell | 0.92 |
| | carcinoma | |

*Flux determined using an in vitro method described in Example 1.
**Estimated flux based on known potency relative to lidocaine.

The therapeutically effective flux values in Table A (with the exception of ropivacaine) represent the steady state flux values of marketed products through hairless mouse or human epidermal membrane in an in vitro system described in Example 1. These values are meant only to be estimates and to provide a basis of comparison for formulation development and optimization. The therapeutically effective flux for a selected drug could be very different for different diseases to be treated for, different stages of diseases, and different individual subjects. It should be noted that the flux listed may be more than therapeutically effective.

The following examples listed in Table B illustrate screening of non-volatile solvent's flux enabling ability for some of the drugs specifically studied.

Experiments were carried out as described in Example 1 below and the results are further discussed in the subsequent Examples 2-9.

TABLE B
In vitro steady state flux values of various drugs from non-volatile
solvent systems

| | | Average Flux* |
|---|---|---|
| Drug | Non-Volatile Solvent | (mcg/cm²/hr) |
| Betamethasone | Oleic acid | 0.009 ± 0.003 |
| Dipropionate | Sorbitan Monolaurate | 0.03 ± 0.02 |
| Clobetasol | Propylene Glycol (PG) | 0.0038 ± 0.0004 |
| Propionate | Light Mineral Oil | 0.031 ± 0.003 |
| | Isostearic acid (ISA) | 0.019 ± 0.003 |
| Ropivacaine | Glycerol | 1.2 ± 0.7 |
| | Mineral Oil | 8.9 ± 0.6 |
| Ketoprofen | Polyethylene glycol | 5 ± 2 |
| | 400 | |
| | Span 20 | 15 ± 3 |
| Acyclovir | Polyethylene glycol | 0 |
| | 400 | |
| | Isostearic acid + 10% | 2.7 ± 0.6 |
| | trolamine | |

*Each value represents the mean and st. dev of three determinations.

The in vitro steady state flux values in Table B from non-volatile solvents show surprising flux-enabling and non flux-enabling solvents. This information can be used to guide formulation development.

The term “plasticizing” in relation to flux-enabling non-volatile solvent(s) is defined as a flux-enabling non-volatile solvent that acts as a plasticizer for the solidifying agent. A “plasticizer” is an agent which is capable of increasing the percentage elongation of the formulation after the volatile solvent system has at least substantially evaporated. Plasticizers also have the capability to reduce the brittleness of solidified formulation by making it more flexible and/or elastic. For example, propylene glycol is a “flux-enabling, plasticizing non-volatile solvent” for the drug ketoprofen with polyvinyl alcohol as the selected solidifying agent. However, propylene glycol in a formulation of ketoprofen with Gantrez S-97 or Avalure UR 405 as solidifying agents does not provide the same plasticizing effect. The combination of propylene glycol and Gantrez S-97 or Avalure UR 405 is less compatible and results in less desirable formulation for topical applications. Therefore, whether a given non-volatile solvent is “plasticizing” depends on which solidifying agent(s) is selected.

Different drugs often have different matching flux-enabling non-volatile solvent systems which provide particularly good results. Examples of such are noted in Table C. Experiments were carried out as described in Example 1 below and the results are further discussed in the subsequent Examples 2-9.

TABLE C
In vitro steady state flux values of various drugs from particularly high
flux-enabling non-volatile solvent systems

| | High flux-enabling non- | Avg. Flux* |
|---|---|---|
| Drug | volatile solvent | (mcg/cm²/h) |
| Ropivacaine | ISA | 11 ± 2 |
| | Span 20 | 26 ± 8 |
| Ketoprofen | Propylene glycol (PG) | 90 ± 50 |
| Acycolvir | ISA + 30% trolamine | 7 ± 2 |
| Betamethasone | Propylene Glycol | 0.20 ± 0.07 |
| Dipropionate | | |
| Clobetasol | PG + ISA (Ratio of PG:ISA | 0.8 ± 0.2 |
| Propionate | ranging from 200:1 to 1:1) | |

*Each value represents the mean and st. dev of three determinations.

It should be noted that “flux-enabling non-volatile solvent,” “flux-enabling, plasticizing non-volatile solvent,” or “high flux-enabling non-volatile solvent” can be a single chemical substance or a mixture of two or more chemical substances. For example, the steady state flux value for clobetasol propionate in Table C is a 9:1 for propylene glycol:isostearic acid mixture that generated much higher clobetasol flux than propylene glycol or ISA alone (see Table B). Therefore, the 9:1 propylene glycol:isostearic acid mixture is a “high flux-enabling non-volatile solvent” but propylene glycol or isostearic acid alone is not.

The term “adhesion” or “adhesive” when referring to a solidified layer herein refers to sufficient adhesion between the solidified layer and the skin so that the layer does not fall off the skin during intended use on most subjects. Thus, “adhesive” or the like when used to describe the solidified layer means the solidified layer is adhesive to the body surface to which the initial formulation layer was originally applied (before the evaporation of the volatile solvent(s)). In one embodiment, it does not mean the solidified layer is adhesive on the opposing side. In addition, it should be noted that whether a solidified layer can adhere to a skin surface for the desired extended period of time partially depends on the condition of the body surface. For example, excessively sweating or oily skin, or oily substances on the skin surface may make the solidified layer less adhesive to the skin. Therefore, the adhesive solidified layer of the current invention may not be able to maintain perfect contact with the body surface and deliver the drug over a sustained period of time for every subject under any conditions on the body surface. A standard is that it maintains good contact with most of the body surface, e.g. 70% of the total area, over the specified period of time for most subjects under normal conditions of the body surface and external environment.

The terms “flexible,” “elastic,” “elasticity,” or the like, as used herein refer to sufficient elasticity of the solidified layer so that it is not broken if it is stretched in at least one direction by up to about 5%, and often to about 10% or even greater. For example, a solidified layer that exhibits acceptably elasticity and adhesion to skin can be attached to human skin over a flexible skin location, e.g., elbow, finger, wrist, neck, lower back, lips, knee, etc., and will remain substantially intact on the skin upon stretching of the skin. It should be noted that the solidified layers of the present invention do not necessarily have to have any elasticity in some embodiments.

The term “peelable,” when used to describe the solidified layer, means the solidified layer can be lifted from the skin surface in one large piece or few to several large pieces, as opposed to many small pieces or crumbs.

The term “sustained” relates to therapeutically effective rates of dermal drug delivery for a continuous period of time of at least 30 minutes, and in some embodiments, periods of time of at least about 2 hours, 4 hours, 8 hours, 12 hours, 24 hours, or longer.

“Volatile solvent system” can be a single solvent or a mixture of solvents that are volatile, including water and solvents that are more volatile than water. Non-limiting examples of volatile solvents that can be used in the present invention include denatured alcohol, methanol, ethanol, isopropyl alcohol, water, propanol, C4-C6 hydrocarbons, butane, isobutene, pentane, hexane, acetone, ethyl acetate, fluoro-chloro-hydrocarbons, methyl ethyl ketone, methyl ether, hydrofluorocarbons, ethyl ether, 1,1,1,2 tetrafluorethane 1,1,1,2,3,3,3-heptafluoropropane, 1,1,1,3,3,3 hexafluoropropane, or combinations thereof.

“Non-volatile solvent system” can be a single solvent or mixture of solvents that are less volatile than water. It can also contain substances that are solid or liquid at room temperatures, such as pH or ion-pairing agents. After evaporation of the volatile solvent system, most of the non-volatile solvent system should remain in the solidified layer for an amount of time sufficient to dermally delivery a given drug to, into, or through the skin of a subject at a sufficient flux for a period of time to provide a therapeutic effect. In some embodiments, in order to obtain desired permeability for an active drug and/or compatibility with solidifying agents or other ingredients of the formulation, a mixture of two or more non-volatile solvents can be used to form the non-volatile solvent system. In one embodiment, the combination of two or more non-volatile solvents to form a solvent system provides a higher transdermal flux for a drug than the flux provided for the drug by each of the non-volatile solvents individually. The non-volatile solvent system may also serve as a plasticizer of the solidified layer, so that the solidified layer is elastic and flexible.

The term “solvent vehicle” describes compositions that include both a volatile solvent system and non-volatile solvent system. The volatile solvent system is chosen so as to evaporate from the adhesive formulation quickly to form a solidified layer, and the non-volatile solvent system is formulated or chosen to substantially remain as part of the solidified layer after volatile solvent system evaporation so as to provide continued delivery of the drug. Typically, the drug can be partially or completely dissolved in the solvent vehicle or formulation as a whole. Likewise, the drug can also be partially or completely solubilizable in the non-volatile solvent system once the volatile solvent system is evaporated. Formulations in which the drug is only partially dissolved in the non-volatile solvent system after the evaporation of the volatile solvent system have the potential to maintain longer duration of sustained delivery, as the undissolved drug can dissolve into the non-volatile solvent system as the dissolved drug is being depleted from the solidified layer during drug delivery.

“Adhesive solidifying formulation” or “solidifying formulation” refers to a composition that has a viscosity suitable for application to a skin surface prior to evaporation of its volatile solvent(s), and which can become a solidified layer after evaporation of at least a portion of the volatile solvent(s). The solidified layer, once formed, can be very durable. In one embodiment, once solidified on a skin surface, the formulation can form a peel. The peel can be a soft, coherent solid that can be removed by peeling large pieces from the skin relative to the size of the applied formulation, and often, can be peeled from the skin as a single piece. The application viscosity is typically more viscous than a water-like liquid, but less viscous than a soft solid. Examples of preferred viscosities include materials that have consistencies similar to pastes, gels, ointments, and the like, e.g., viscous liquids that flow but are not subject to spilling. Thus, when a composition is said to have a viscosity “suitable for application” to a skin surface, this means the composition has a viscosity that is high enough so that the composition does not substantially run off the skin after being applied to skin, but also has a low enough viscosity so that it can be easily spread onto the skin. A viscosity range that meets this definition can be from about 100 cP to about 3,000,000 cP (centipoises), and more preferably from about 1,000 cP to about 1,000,000 cP.

In some embodiments of the present invention, it may be desirable to add an additional agent or substance to the formulation so as to provide enhanced or increased adhesive characteristics. The additional adhesive agent or substance can be an additional non-volatile solvent or an additional solidifying agent. Non-limiting examples of substances which might be used as additional adhesion enhancing agents include copolymers of methylvinyl ether and maleic anhydride (Gantrez polymers), polyethylene glycol and polyvinyl pyrrolidone, gelatin, low molecular weight polyisobutylene rubber, copolymer of acrylsan alkyl/octylacrylamido (Dermacryl 79), and/or various aliphatic resins and aromatic resins.

The terms “washable,” “washing” or “removed by washing” when used with respect to the adhesive formulations of the present invention refers to the ability of the adhesive formulation to be removed by the application of a washing solvent using a normal or medium amount of washing force. The required force to remove the formulations by washing should not cause significant skin irritation or abrasion. Generally, gentle washing force accompanied by the application of an appropriate washing solvent is sufficient to remove the adhesive formulations disclosed herein. The solvents which can be used for removing by washing the formulations of the present invention are numerous, but preferably are chosen from commonly acceptable solvents including the volatile solvents listed herein. Preferred washing solvents do not significantly irritate human skin and are generally available to the average subject. Examples of washing solvents include but are not limited to water, ethanol, methanol, isopropyl alcohol, acetone, ethyl acetate, propanol, or combinations thereof. In aspect of the invention the washing solvents can be selected from the group consisting of water, ethanol, isopropyl alcohol, or combinations thereof. Surfactants can also be used in some embodiments.

An acceptable length of time for “drying time” refers to the time it takes for the formulation to form a non-messy solidified surface after application on skin under standard skin and ambient conditions, and with standard testing procedure. It is noted that the word “drying time” as used herein does not mean the time it takes to completely evaporate off the volatile solvent(s). Instead, it means the time it takes to form the non-messy solidified surface as described above.

“Standard skin” is defined as dry, healthy human skin with a surface temperature of between about 30° C. to about 36° C. Standard ambient conditions are defined by the temperature range of from 20° C. to 25° C. and a relative humidity range of from 20% to 80%. The term “standard skin” in no way limits the types of skin or skin conditions on which the formulations of the present invention can be used. The formulations of the present invention can be used to treat all types of “skin,” including undamaged (standard skin), diseased skin, or damaged skin. Although skin conditions having different characteristics can be treated using the formulations of the present invention, the use of the term “standard skin” is used merely as a standard to test the compositions of the varying embodiments of the present invention. As a practical matter, formulations that perform well (e.g., solidify, provide therapeutically effective flux, etc.) on standard skin can also perform well diseased or damaged skin.

The “standard testing procedure” or “standard testing condition” is as follows: to standard skin at standard ambient conditions is applied an approximately 0.1 mm layer of the adhesive solidifying formulation and the drying time is measured. The drying time is defined as the time it takes for the formulation to form a non-messy surface such that the formulation does not lose mass by adhesion to a piece of 100% cotton cloth pressed onto the formulation surface with a pressure of between about 5 and about 10 g/cm² for 5 seconds.

“Solidified layer” describes the solidified or dried layer of an adhesive solidifying formulation after at least a portion of the volatile solvent system has evaporated. The solidified layer remains adhered to the skin, and is preferably capable of maintaining good contact with the subject's skin for substantially the entire duration of application under standard skin and ambient conditions. The solidified layer also preferably exhibits sufficient tensile strength so that it can be peeled off the skin at the end of the application in one piece or several large pieces (as opposed to a layer with weak tensile strength that breaks into many small pieces or crumbles when removed from the skin).

The use of the term “substantially” when referring to the evaporation of the volatile solvents means that a majority of the volatile solvents which were included in the initial formulation have evaporated. Similarly, when a solidified layer is said to be “substantially devoid” of volatile solvents, including water, the solidified layer has less than 10 wt %, and preferably less than 5 wt %, of the volatile solvents in the solidified layer as a whole.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 0.01 to 2.0 mm” should be interpreted to include not only the explicitly recited values of about 0.01 mm to about 2.0 mm, but also include individual values and sub-ranges within the indicated range. Thus, included in this numerical range are individual values such as 0.5, 0.7, and 1.5, and sub-ranges such as from 0.5 to 1.7, 0.7 to 1.5, and from 1.0 to 1.5, etc. This same principle applies to ranges reciting only one numerical value. Furthermore, such an interpretation should apply regardless of the breadth of the range or the characteristics being described.

As used herein, a plurality of drugs, compounds, and/or solvents may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

With these definitions in mind, the present invention is drawn generally to a formulation for treating an infection, comprising a drug that is effective for treating an infection, a solvent vehicle, and a solidifying agent. The solvent vehicle can comprise a volatile solvent system including at least one volatile solvent, and a non-volatile solvent system including at least one non-volatile solvent, wherein the non-volatile solvent system is capable of facilitating delivery of the drug at therapeutically effective rates over a sustained period of time. The formulation can have a viscosity suitable for application and adhesion to a skin surface prior to evaporation of the volatile solvent system. The formulation applied to the skin surface can form a solidified layer after at least partial evaporation of the volatile solvent system. Further, the drug can continue to be delivered after the volatile solvent system is at least substantially evaporated.

In another embodiment, a method of treating a skin infection can comprise applying a solidifying adhesive formulation to an infected skin surface. The solidifying adhesive formulation can comprise a drug that is effective for treating a skin infection, a solvent vehicle, and a solidifying agent. The solvent system can comprise a volatile solvent system including at least one volatile solvent, and a non-volatile solvent system including at least one non-volatile solvent. The non-volatile solvent system can be capable of facilitating the delivery of the drug at therapeutically effective rates over a sustained period of time. The formulation can have a viscosity suitable for application and adhesion to the skin surface prior to evaporation of the volatile solvent system. Additional steps include solidifying the formulation to form a solidified layer on the infected skin surface by at least partial evaporation of the volatile solvent system, and dermally delivering the drug from the solidified layer to the infected skin site at therapeutically effective rates over a sustained period of time.

In another embodiment, a solidified layer for treating an infection can comprise a drug that is effective for treating a skin infection; a non-volatile solvent system including at least one non-volatile solvent, wherein the non-volatile solvent system facilitates the delivery of the drug at therapeutically effective rates over a sustained period of time; and a solidifying agent. The solidified layer can be stretchable by 5% (or even 10%) in one direction without cracking, breaking, and/or separating from a skin surface to which the layer is applied.

In still another embodiment, a formulation for treating an infection can comprise a drug selected from the group consisting of acyclovir, valacyclovir, pencyclovir, or combinations thereof; a solvent vehicle comprising a volatile solvent system including at least one volatile solvent, and a non-volatile solvent system comprising a non-volatile solvent; and a solidifying agent. The non-volatile solvent can be selected from the group consisting of oleic acid, isostearic acid, olive oil, or combinations thereof. The solidifying agent can be selected from the group consisting of ethyl acrylate-methyl methacrylate-trimethylammonioethyl methacrylate chloride copolymers, butyl and methyl methacrylate copolymers, ethyl cellulose, and mixtures and copolymers thereof. The formulation can have a viscosity suitable for application to a skin surface prior to evaporation of the volatile solvent system, can form a solidified, coherent, flexible, and continuous layer after at least partial evaporation of the volatile solvent system, and the drug can be continued to be delivered at a therapeutically effective rate after the volatile solvent system is at least substantially all evaporated.

In another embodiment, a formulation for treating an infection can comprise a drug selected from the group consisting of econazole, terbinafine, or combinations thereof; a solvent vehicle comprising a volatile solvent system including at least one volatile solvent and a non-volatile solvent system comprising at least one non-volatile solvent, and a solidifying agent. The non-volatile solvent can be selected from the group consisting of tetrahydroxypropyl ethylenediamine, oleic acid, isostearic acid, olive oil, or combinations thereof. The solidifying agent can be selected from the group consisting of ethyl acrylate-methyl methacrylate-trimethylammonioethyl methacrylate chloride copolymers, butyl and methyl methacrylate copolymers, ethyl cellulose, and mixtures and copolymers thereof. The formulation can have a viscosity suitable for application to a skin surface prior to evaporation of the volatile solvent system, can form a solidified, coherent, flexible, and continuous layer after at least partial evaporation of the volatile solvent system, and the drug can be continued to be delivered at a therapeutically effective rate after the volatile solvent system is at least substantially all evaporated.

In another embodiment, an adhesive solidifying formulation for treating a nail infection can comprise a drug that is effective for treating a nail infection, a solvent vehicle, and a solidifying agent. The solvent vehicle can comprise a volatile solvent system including at least one volatile solvent, and a non-volatile solvent system including at least one non-volatile solvent, wherein the non-volatile solvent system is capable of facilitating delivery of the drug at a therapeutically effective rate over a sustained period of time. The formulation has a viscosity suitable for application and adhesion to a nail surface prior to evaporation of the volatile solvent system, and when applied to the nail surface, it forms a solidified layer after at least partial evaporation of the volatile solvent system. Further, the drug continues to be delivered to the nail after the volatile solvent system is at least substantially evaporated.

In another embodiment, a method of treating nail fungal infection can comprise applying to a nail surface with a fungal infection, and optionally surrounding skin, a layer of an adhesive solidifying formulation. The formulation can comprise an anti-fungal drug, a solvent vehicle including a volatile solvent system comprising at least one volatile solvent, and a non-volatile solvent system comprising at least one non-volatile solvent, and a solidifying agent. The non-volatile solvent system can be capable of facilitating delivery of the anti-fungal drug at a therapeutically effective rate over a sustained period of time, and can have a viscosity suitable for application and adhesion to a nail surface prior to evaporation of the volatile solvent system. Further, the formulation applied to the nail surface can form a solidified layer after at least partial evaporation of the volatile solvent system, and the drug can continue to be delivered from the solidified layer to the nail after the volatile solvent system is at least substantially evaporated. Additional steps can include keeping the solidified layer on said nail surface for a treatment period of at least 4 hours, and removing the solidified layer after the treatment period.

Thus, the present invention is related to formulations that are typically in the initial form of semi-solids (including creams, gels, pastes, ointments, and other viscous liquids), which can be easily applied onto the skin as a layer, and can, after evaporation of at least some of the volatile solvent(s), quickly (from 15 seconds to about 5 minutes under standard skin and ambient conditions as set forth above) to moderately quickly (from about 4 to about 15 minutes under standard skin and ambient conditions) change into a solidified layer (which is optionally also peelable), e.g., a coherent and soft solid layer, for drug delivery. The solidified layer thus formed is capable of delivering drug over a sustained period of time, e.g., hours to tens of hours, so that most of the drug absorption occurs after the solidified layer is formed.

Additionally, the solidified layer typically adheres to the skin, but has a solidified, minimally-adhering, outer surface which is formed relatively soon after application and which does not substantially transfer to or otherwise soil clothing or other objects that a subject is wearing or that the solidified layer may inadvertently contact. The solidified layer can also be formulated such that it is highly flexible and stretchable, and thus, is capable of maintaining good contact with a skin surface, even if the skin is stretched during normal daily activities.

The formulations of the present invention can be applied and used on various types of human body or skin surfaces. In one embodiment, the skin surface being treated can be what is traditionally referred to as “skin.” The skin surface can be an epidermal layer of the skin. In another embodiment, the skin surface that can be treated is a mucosal surface, such as lips, oral mucosal, genital mucosa, nasal mucosa, or anal mucosa. In another embodiment, the skin surface being treated can be a finger or toe nail surface. In yet another embodiment, the skin surface being treated is a wounded skin surface. In yet another embodiment, the skin surface is a bed sore or a skin surface with one or more lesions or open sores.

In selecting the various components that can be used, e.g., drug, solvent vehicle of volatile solvent system and non-volatile solvent system, solidifying agent(s), etc., many variations can be considered. For example, the volatile solvent system may be one or more volatile solvents (at least as volatile as water, including water). In one embodiment of the present invention, the volatile solvent system can include a member of ethanol, isopropyl alcohol, water, dimethyl ether, diethyl ether, butane, propane, isobutene, 1,1, difluoroethane, 1,1,1,2 tetrafluorethane, 1,1,1,2,3,3,3-heptafluoropropane, 1,1,1,3,3,3 hexafluoropropane, ethyl acetate, acetone, or combinations thereof. In another embodiment of the present invention, the volatile solvent system can include denatured alcohol, methanol, propanol, isobutene, pentane, hexane, cytopentasiloxane, cyclomethicone, methyl ethyl ketone, or combinations thereof. The volatile solvent system can include a mixture or combination of any of the volatile solvents set forth in the embodiments above. These volatile solvents should be chosen to be compatible with the rest of the formulation. It is desirable to use an appropriate weight percentage of the volatile solvent(s) in the formulation. Too much of the volatile solvent system prolongs the drying time. Too little of the volatile solvent system can make it difficult to spread the formulation on the skin. For most formulations, the weight percentage of the volatile solvent(s) can be from about 10 wt % to about 85 wt %, and more preferably from about 20 wt % to about 50 wt %.

The non-volatile solvent system can also be chosen or formulated to be compatible with the solidifying agent, the drug, the volatile solvent, and any other ingredients that may be present. For example, the solidifying agent can be chosen so that it is dispersible or soluble in the non-volatile solvent system. Most non-volatile solvent systems and solvent vehicles as a whole can be formulated appropriately after experimentation. For instance, certain drugs have good solubility in poly ethylene glycol (PEG) having a molecular weight of 400 (PEG 400, non-volatile solvent) but poor solubility in glycerol (non-volatile solvent) and water (volatile solvent). However, PEG 400 cannot effectively dissolve poly vinyl alcohol (PVA), and thus, is not very compatible alone with PVA as the only solidifying agent. In order to dissolve sufficient amount of an active drug and use PVA as a solidifying agent at the same time, a non-solvent system including PEG 400 and glycerol (compatible with PVA) in an appropriate ratio can be formulated, achieving a compatibility compromise. As a further example of compatibility, non-volatile solvent/solidifying agent incompatibility is observed when Span 20 is formulated into a formulation containing PVA. With this combination, Span 20 can separate out of the formulation and form an oily layer on the surface of the solidified layer. Thus, appropriate solidifying agent/non-volatile solvent selections are desirable in developing a viable formulation and compatible combinations.

Non-volatile solvent(s) that can be used alone or in combination to form non-volatile solvent systems can be selected from a variety of pharmaceutically acceptable liquids. In one embodiment of the present invention, the non-volatile solvent system can include glycerol, propylene glycol, isostearic acid, oleic acid, propylene glycol, trolamine, tromethamine, triacetin, sorbitan monolaurate, sorbitan monooleate, sorbitan monopalmitate, or combinations thereof. In another embodiment the non-volatile solvent system can include benzoic acid, dibutyl sebecate, diglycerides, dipropylene glycol, eugenol, fatty acids such as coconut oil, fish oil, palm oil, grape seed oil, isopropyl myristate, mineral oil, oleyl alcohol, vitamin E, triglycerides, sorbitan fatty acid surfactants, triethyl citrate, or combinations thereof. In a further embodiment, the non-volatile solvent system can include 1,2,6-hexanetriol, alkyltriols, alkyldiols, tocopherol, p-propenylanisole, anise oil, apricot oil, dimethyl isosorbide, alkyl glucoside, benzyl alcohol, bees wax, benzyl benzoate, butylene glycol, caprylic/capric triglyceride, caramel, cassia oil, castor oil, cinnamaldehyde, cinnamon oil, clove oil, coconut oil, cocoa butter, cocoglycerides, coriander oil, corn oil, corn syrup, cottonseed oil, cresol, diacetin, diacetylated monoglycerides, diethanolamine, diglycerides, ethylene glycol, eucalyptus oil, fat, fatty alcohols, flavors, liquid sugars ginger extract, glycerin, high fructose corn syrup, hydrogenated castor oil, IP palmitate, lemon oil, lime oil, limonene, monoacetin, monoglycerides, nutmeg oil, octyldodecanol, orange oil, palm oil, peanut oil, PEG vegetable oil, peppermint oil, petrolatum, phenol, pine needle oil, polypropylene glycol, sesame oil, spearmint oil, soybean oil, vegetable oil, vegetable shortening, wax, 2-(2-(octadecyloxy)ethoxy)ethanol, benzyl benzoate, butylated hydroxyanisole, candelilla wax, carnauba wax, ceteareth-20, cetyl alcohol, polyglyceryl, dipolyhydroxy stearate, PEG-7 hydrogenated castor oil, diethyl phthalate, diethyl sebacate, dimethicone, dimethyl phthalate, PEG Fatty acid esters such as PEG-stearate, PEG-oleate, PEG-laurate, PEG fatty acid diesters such as PEG-dioleate, PEG-distearate, PEG-castor oil, glyceryl behenate, PEG glycerol fatty acid esters such as PEG glyceryl laurate, PEG glyceryl stearate, PEG glyceryl oleate, lanolin, lauric diethanolamide, lauryl lactate, lauryl sulfate, medronic acid, multisterol extract, myristyl alcohol, neutral oil, PEG-octyl phenyl ether, PEG-alkyl ethers such as PEG-cetyl ether, PEG-stearyl ether, PEG-sorbitan fatty acid esters such as PEG-sorbitan diisosterate, PEG-sorbitan monostearate, propylene glycol fatty acid esters such as propylene glycol stearate, propylene glycol, caprylate/caprate, sodium pyrrolidone carboxylate, sorbitol, squalene, stear-o-wet, triglycerides, alkyl aryl polyether alcohols, polyoxyethylene derivatives of sorbitan-ethers, saturated polyglycolyzed C8-C10 glycerides, N-methylpyrrolidone, honey, polyoxyethylated glycerides, dimethyl sulfoxide, azone and related compounds, dimethylformamide, N-methyl formamaide, fatty acid esters, fatty alcohol ethers, alkyl-amides (N,N-dimethylalkylamides), N-methylpyrrolidone related compounds, ethyl oleate, polyglycerized fatty acids, glycerol monooleate, glyceryl monomyristate, glycerol esters of fatty acids, silk amino acids, PPG-3 benzyl ether myristate, Di-PPG2 myreth 10-adipate, honeyquat, sodium pyroglutamic acid, abyssinica oil, dimethicone, macadamia nut oil, limnanthes alba seed oil, cetearyl alcohol, PEG-50 shea butter, shea butter, aloe vera juice, phenyl trimethicone, hydrolyzed wheat protein, or combinations thereof. In yet a further embodiment the non-volatile solvent system can include a combination or mixture of non-volatile solvents set forth in the any of the above discussed embodiments.

Certain volatile and/or non-volatile solvent(s) that are irritating to the skin may be desirable to use to achieve the desired solubility and/or permeability of the drug. It is also desirable to add compounds that are both capable of preventing or reducing skin irritation and are compatible with the formulation. For example, in a formulation where the solvent (either non-volatile or volatile) is capable of irritating the skin, it would be helpful to use a non-volatile solvent that is capable of reducing skin irritation. Examples of solvents that are known to be capable of preventing or reducing skin irritation include, but are not limited to, glycerin, honey, and propylene glycol, although other irritation reducing solvents may also be used.

The formulations of the current invention may also contain two or more non-volatile solvents that independently are not adequate non-volatile solvents for a drug but when formulated together become an adequate non-volatile solvent. One possible reason for these initially non adequate non-volatile solvents to become adequate non-volatile solvents when formulated together may be due to the optimization of the ionization state of the drug to a physical form which has higher flux or the non-volatile solvents act in some other synergistic manner. One further benefit of the mixing of the non-volatile solvents is that it may optimize the pH of the formulation or the skin tissues under the formulation layer to minimize irritation. Examples of suitable combinations of non-volatile solvents that result in an adequate non-volatile solvent system include but are not limited to isostearic acid/trolamine, isostearic acid/diisopropyl amine, oleic acid/trolamine, and propylene glycol/isostearic acid.

The selection of the solidifying agent can also be carried out in consideration of the other components present in the solidifying adhesive formulation. An appropriate solidifying agent is compatible with the formulation such that the formulation is in liquid or semi-liquid state, e.g. cream, paste, gel, ointment, etc., before any evaporation of the volatile solvent(s) and becomes a soft, coherent solid after the evaporation of at least some of the volatile solvent(s). The solidifying agent can be selected or formulated to be compatible with the drug and the solvent vehicle (including the volatile solvent(s) and the non-volatile solvent system), as well as provide desired physical properties to the solidified layer once it is formed. Depending on the drug, solvent vehicle, and/or other components that may be present, the solidifying agent can be selected from a variety of agents. In one embodiment, the solidifying agent can include polyvinyl alcohol with a MW range of 20,000-70,000 (Amresco), esters of polyvinylmethylether/maleic anhydride copolymer (ISP Gantrez ES-425 and Gantrez ES-225) with a MW range of 80,000-160,000, neutral copolymer of butyl methacrylate and methyl methacrylate (degussa Plastoid B) with a MW range of 120,000-180,000, dimethylaminoethyl methacrylate-butyl methacrylate-methyl methacrylate copolymer (degussa Eudragit E100) with a MW range of 100,000-200,000, ethyl acrylate-methyl methacrylate-trimethylammonioethyl methacrylate chloride copolymer with a MW greater than 5,000 or similar MW to Eudragit RLPO (Degussa), Zein (prolamine) with a MW greater than 5,000 (Zein, MW around 35,000, Freeman industries), pregelatinized starch having a MW similar to Instant Pure-Cote B793 (Grain Processing Corporation), ethyl cellulose with a MW greater than 5,000 or a MW similar to Aqualon EC N7, N10, N14, N22, N50, or N100 (Hercules), fish gelatin having a MW range of 20,000-250,000 (Norland Products), gelatin, other animal sources with a MW range greater than 5,000, acrylates/octylacrylamide copolymer with a MW range greater than 5,000 or a MW similar to National Starch and Chemical Dermacryl 79.

In another embodiment, the solidifying agent can include ethyl cellulose, hydroxy ethyl cellulose, hydroxy methyl cellulose, hydroxy propyl cellulose, hydroxypropyl methyl cellulose, carboxymethyl cellulose, methyl cellulose, polyether amides, corn starch, pregelatinized corn starch, polyether amides, shellac, polyvinyl pyrrolidone, polyisobutylene rubber, polyvinyl acetate phthalate, or combinations thereof. In a further embodiment, the solidifying agent can include ammonia methacrylate, carrageenan, cellulose acetate phthalate aqueous such as CAPNF from Eastman, carboxy polymethylene, cellulose acetate (microcrystalline), cellulose polymers, divinyl benzene styrene, ethylene vinyl acetate, silicone, guar gum, guar rosin, gluten, casein, calcium caseinate, ammonium caseinate, sodium caseinate, potassium caseinate, methyl acrylate, microcrystalline wax, polyvinyl acetate, PVP ethyl cellulose, acrylate, PEG/PVP, xantham gum, trimethyl siloxysilicate, maleic acid/anhydride colymers, polacrilin, poloxamer, polyethylene oxide, poly glactic acid/poly-I-lactic acid, turpene resin, locust bean gum, acrylic copolymers, polyurethane dispersions, dextrin, polyvinyl alcohol-polyethylene glycol co-polymers, methyacrylic acid-ethyl acrylate copolymers such as BASF's Kollicoat polymers, methacrylic acid and methacrylate based polymers such as poly(methacrylic acid), or combinations thereof. In yet a further embodiment, the solidifying agent can include a combination of solidifying agents set forth in the any of the above discussed embodiments. Other polymers may also be suitable as the solidifying agent, depending on the solvent vehicle components, the drug, and the specific functional requirements of the given formulation.

In one embodiment of the present invention, the solidifying agent includes a methacrylic polymer or copolymer such as methyacrylic acid-ethyl acrylate copolymer, butyl and methyl methacrylate copolymer, aminoalkyl methacrylate copolymer, and/or an ammonioalkyl methacrylate copolymer. In another embodiment, the solidifying agent includes polyvinyl alcohol or a polyvinyl alcohol copolymer such as polyvinyl alcohol-polyethylene glycol copolymer.

The non-volatile solvent system and the solidifying agent are preferably compatible with one other. Compatibility can be defined as i) the solidifying agent does not substantially negatively influence the function of the non-volatile solvent system; ii) the solidifying agent can hold the non-volatile solvent system in the solidified layer so that substantially no non-volatile solvent oozes out of the layer, and iii) the solidified layer formed with the selected non-volatile solvent system and the solidifying agent has acceptable flexibility, rigidity, tensile strength, elasticity, and adhesiveness. The weight ratio of the non-volatile solvent system to the solidifying agent can be from about 0.1:1 to about 10:1, or more preferably from about 0.5:1 to about 2:1. In some embodiments, the non-volatile solvent system makes up about 20-60% of the total weight of the formulation,

The thickness of the formulation layer applied on the skin should also be appropriate for a given formulation and desired drug delivery considerations. If the layer is too thin, the amount of the drug may not be sufficient to support sustained delivery over the desired length of time. If the layer is too thick, it may take too long to form a non-messy outer surface of the solidified layer. If the drug is very potent and the solidified layer has very high tensile strength, a layer as thin as 0.01 mm may be sufficient. If the drug has rather low potency and the solidified layer has low tensile strength, a layer as thick as 2-3 mm may be needed. Thus, for most drugs and formulations, the appropriate thickness can be from about 0.01 mm to about 3 mm, but more typically, from about 0.05 mm to about 1 mm.

The flexibility and stretchability of a solidified layer, optionally in the form of a peel, can be desirable in some applications. High flex and stretch are particularly advantageous when the area being treated is involved in frequent stretching or movement, such as the lips or corners of the mouth. Traditional ointments, creams, gels, pastes or the like are often not suitable for treatment of these areas because they are easily removed by licking the lips or through contact with food during eating. In contrast, the solidifying compositions of the present invention can be formulated so as to provide adequate flexibility and stretching while not being easily licked, rubbed, or scraped off. It is also worth noting that the solidified layers of the present invention do not always need to be stretchable, though some elasticity is preferred.

A further feature of a formulation is related to the drying time. If a formulation dries too quickly, the user may not have sufficient time to spread the formulation into a thin layer on the skin surface before the formulation is solidified, leading to poor skin contact. If the formulation dries too slowly, the subject may have to wait a long time before resuming normal activities (e.g. putting clothing on, eating, talking, etc) that may remove un-solidified formulation. Thus, it is desirable for the drying time to be longer than about 15 seconds but shorter than about 15 minutes, and preferably from about 0.5 minutes to about 5 minutes.

Other benefits of the solidified layers of the present invention include the presence of a physical barrier that can be formed by the material itself. This physical barrier can protect the infected area against contacting objects or sources which cause irritation, pain, or further infections. For example, the solidified layer can act as a barrier against friction with a diaper, or as a protective barrier against urine and/or fecal matter. Additionally, upon volatile solvent system evaporation, the dosage form is relatively thick and can contain much more active drug than a typical layer of traditional cream, gel, lotion, ointment, paste, etc., and further, is not as subject to unintentional removal.

These and other advantages can be summarized by the following non-limiting application embodiments. The solidified layers of the present invention can be prepared in an initial form that is easy to apply as a semisolid dosage form. Additionally, upon volatile solvent evaporation, the formulation layer applied to the skin is relatively thick and can contain much more active drug than a typical layer of traditional cream, gel, lotion, ointment, paste, etc., and further, is resistant to unintentional removal. After the evaporation of the volatile solvent(s) and the formation of the solidified layer, the drug in the solidified layer can be delivered at therapeutically effective rates over sustained periods of time. Further, as the solidified layer remains adhesive to skin and, easy removal of the solidified layer can occur, usually without the aid of a solvent or surfactant. In some embodiments, the adhesion to skin and elasticity of the material is such that the solidified layer will not separate from the skin upon skin stretching at highly stretchable skin areas, such as over joints and muscles. For example, in one embodiment, the solidified layer can be stretched by 5% or even 10% or greater in at least one direction without cracking, breaking, and/or separating form a skin surface to which the layer is applied.

As a further note, it is a unique feature that the solidified layers of the present invention can keep a substantial amount of the non-volatile solvent system, which is optimized for delivering the drug, on the body surface. This feature can provide unique advantages over existing products. For example, Penlac is a product widely used for treating nail fungal infections. It contains the drug ciclopirox, volatile solvents (ethyl acetate and isopropyl), and a polymeric substance. After being applied on the nail surface, the volatile solvents quickly evaporate and the formulation layer solidifies into a hard lacquer. The drug molecules are immobilized in the hard lacquer layer and are substantially unavailable for delivery into the nail. As a result, it is believed that the delivery of the drug is not sustained over a long period of time. As a result, without being bound by any particular theory, it is believed that this is at least one of the reasons why Penlac, while widely used, has an efficacy rate of only about 10%. Conversely, in the solidified layer of the present invention, the drug molecules are quite mobile in the non-volatile solvent system which is in contact with the skin surface, e.g., skin, nail, mucosal, etc., surface, thus ensuring sustained delivery.

### EXAMPLES

The following examples illustrate the embodiments of the invention that are presently best known. However, it is to be understood that the following are only exemplary or illustrative of the application of the principles of the present invention. Numerous modifications and alternative compositions, methods, and systems may be devised by those skilled in the art without departing from the spirit and scope of the present invention. The appended claims are intended to cover such modifications and arrangements. Thus, while the present invention has been described above with particularity, the following examples provide further detail in connection with what are presently deemed to be the most practical and preferred embodiments of the invention.

### Example 1

Hairless mouse skin (HMS) or human epidermal membrane (HEM) is used as the model membranes as noted for the in vitro flux studies described in herein. Hairless mouse skin (HMS) is used as the model membrane for the in vitro flux studies described in herein. Freshly separated epidermis removed from the abdomen of a hairless mouse is mounted carefully between the donor and receiver chambers of a Franz diffusion cell. The receiver chamber is filled with pH 7.4 phosphate buffered saline (PBS). The experiment is initiated by placing test formulations (of Examples 2-5) on the stratum corneum (SC) of the skin sample. Franz cells are placed in a heating block maintained at 37° C. and the HMS temperature is maintained at 35° C. At predetermined time intervals, 800 μL aliquots are withdrawn and replaced with fresh PBS solution. Skin flux (μg/cm²/h) is determined from the steady-state slope of a plot of the cumulative amount of permeation versus time. It is to be noted that human cadaver skin can be used as the model membrane for the in vitro flux studies as well. The mounting of the skin and the sampling techniques used as the same as described above for the HMS studies.

### Example 2

Formulations of acyclovir in various non-volatile solvent systems are evaluated. Excess acyclovir is present.

The transdermal flux of acyclovir from the test formulations through HMS is presented in Table 1 below.

TABLE 1

| | Skin Flux* |
|---|---|
| Non-volatile solvent system | (mcg/cm²/h) |
| Isostearic Acid | 0.1 ± 0.09 |
| Isostearic Acid + 10% Trolamine | 2.7 ± 0.6 |
| Isostearic Acid + 30% Trolamine | 7 ± 2 |
| Olive Oil | 0.3 ± 0.2 |
| Olive Oil + 11% Trolamine | 3 ± 3 |
| Olive Oil + 30% Trolamine | 0.3 ± 0.2 |
| Oleic Acid | 0.4 ± 0.3 |
| Oleic Acid + 10% Trolamine | 3.7 ± 0.5 |
| Oleic Acid + 30% Trolamine | 14 ± 5 |
| Ethyl Oleate | 0.2 ± 0.2 |
| Ethyl Oleate + 10% Trolamine | 0.2 ± 0.2 |

*Skin flux measurements represent the mean and standard deviation of three determinations. Flux measurements reported were determined from the linear region of the cumulative amount versus time plots. The linear region was observed to be between 4-8 hours.

As indicated, significant enhancement of acyclovir skin flux is achieved with isostearic acid or oleic acid mixed with trolamine. Relatively significant flux enhancement (e.g., 10 fold) is observed when trolamine is added to olive oil, oleic acid, and isostearic acid and no appreciable flux enhancement is observed when trolamine is added to ethyl oleate. This surprising result may be the result of an additive or even synergistic enhancement effect of trolamine/fatty acid combination resulting in much higher acyclovir flux values.

### Examples 3-6

Prototype adhesive solidifying formulations are prepared as follows. Several acyclovir solidifying formulations are prepared in accordance with embodiments of the present invention in accordance with Table 2, as follows:

TABLE 2

| Example | 3 | 4 | 5 | 6 |
|---|---|---|---|---|
| | % by weight | | | |
| Ethanol | 21 | 25 | 28 | 29.5 |
| Eudragit RL-PO* | 15 | 18 | 20 | 21.0 |
| Isostearic Acid | 31 | 36 | 39 | 42.0 |
| Trolamine | 30 | 18 | 10 | 4.7 |
| Acyclovir | 3 | 3 | 3 | 2.8 |

*degussa polymer.

In Examples 3-6, the compositions in Table 2 are prepared as follows. Eudragit RL-PO and ethanol are combined in a glass jar and heated with stirring until the RL-PO is dissolved. The isostearic acid and trolamine is added to the RL-PO/ethanol mixture and the mixture is vigorously stirred. Once a uniform mixture is obtained, acyclovir is added to the mixture and the formulation is vigorously mixed.

### Examples 7-8

Two acyclovir adhesive solidifying formulations are prepared in accordance with embodiments of the present invention in accordance with Table 3, as follows:

TABLE 3

| Example | 7 | 8 |
|---|---|---|
| | % by weight | |
| Ethanol | 26 | 21 |
| Eudragit RL-PO | 44 | 15 |
| Isostearic Acid | 26 | 31 |
| Diisopropanol Amine | 2 | — |
| Neutrol TE Polyol | — | 30 |
| Acyclovir | 2 | 3 |

The compositions of Examples 7 and 8 as shown in Table 3 are prepared as follows. Eudragit RL-PO and ethanol are combined in a glass jar and heated with stirring until the RL-PO is dissolved. The isostearic acid and diisopropanol amine or Neutrol TE Polyol (BASF) is added to the RL-PO/ethanol mixture and the mixture is vigorously stirred. Once a uniform mixture is obtained, acyclovir is added to the mixture and the formulation is vigorously mixed.

### Examples 9-10

Two acyclovir solidifying formulations are prepared in accordance with embodiments of the present invention in accordance with Table 4, as follows:

TABLE 4

| Example | 9 | 10 |
|---|---|---|
| | % by weight | |
| Ethanol | 59.6 | 58 |
| Ethyl cellulose | 19.9 | — |
| ECN7* | | |
| Ethyl cellulose | — | 19 |
| ECN100* | | |
| Trolamine | 7.6 | 9 |
| Isostearic Acid | 7.7 | 9 |
| Acyclovir | 5.2 | 5 |

*Hercules Aqualon N type ethyl cellulose.

In Examples 9-10 the compositions in Table 4 are prepared as follows. EC7 or EC100 and ethanol are combined in a glass jar and heated with stirring until the solid cellulose is dissolved. The isostearic acid and trolamine is added to the cellulose/ethanol mixture and the mixture is vigorously stirred. Once a uniform mixture is obtained, acyclovir is added to the mixture and the formulation is vigorously mixed.

### Example 11

The formulations of Examples 3-10 are tested in a hairless mouse skin (HMS) in vitro model described in Example 1. Table 5 shows data obtained using the experimental process outlined above.

TABLE 5
Steady-state flux (J) of Acyclovir through HMS

| | J* | Ratio to |
|---|---|---|
| Formulation | (μg/cm²/h) | Control |
| Example 3 | 12 ± 5 | 6 |
| Example 4 | 19 ± 1 | 8 |
| Example 5 | 8 ± 1 | 4 |
| Example 6 | 1 ± 1 | 0.5 |
| Example 7 | 0.7 ± 0.3 | 0.35 |
| Example 8 | 1 ± 0.9 | 0.5 |
| Example 9 | 2 ± 1 | 1 |
| Example 10 | 19 ± 7 | 8 |
| Zovirax Cream | 2 ± 0.4 | 1 |

*Skin flux measurements represent the mean and standard deviation of three determinations. Flux measurements reported were determined from the linear region of the cumulative amount versus time plots. The linear region was observed to be between 4-8 hours. If experimental conditions allowed the steady state flux would extend beyond the 8 hours measured.

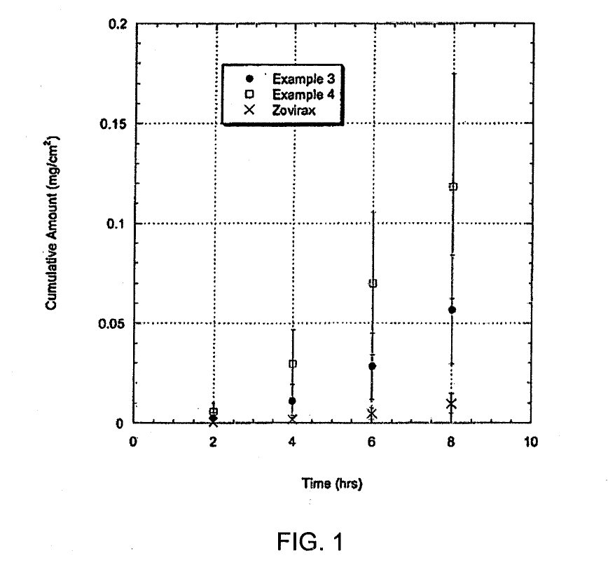

The formulations of the invention shown above generally provide for significant penetration of the active ingredient, and further, the formulations of Examples 3-5 and 10 are found to be much greater in permeability than the marketed product Zovirax Cream (control). The quantity of acyclovir that permeated across the HMS stratum corneum over time for Examples 3, 4, and Zovirax Cream are shown in FIG. 1. Each value shown indicates the mean±SD of at least three experiments.

Examples 3-6 show the impact of the trolamine to isostearic acid (ISA) ratio on acyclovir flux enhancement. The optimal ISA:trolamine ratio is 1:1 to 2:1 and ratio greater than 4:1 show a significant decrease in the acyclovir skin flux. Additions of diisopropanol amine and Neutrol in place of trolamine (Examples 7 and 8) in the formulation show a significant decrease in acyclovir flux values. This may be due to a specific chemical interaction between trolamine and ISA creating an environment within the formulation which facilitates higher skin flux. Examples 9 and 10 utilize a different solidifying agent to evaluate the impact of the solidifying agent on acyclovir flux. Surprisingly, Example 9 shows a significant decrease in acyclovir skin flux, but Example 10, which differed from Example 9 only by the molecular weight of the solidifying agent, shows no impact on acyclovir skin flux compared to a similar ISA:trolamine ratio in Example 3.

As can be seen from FIG. 1, Examples 3 and 4 show sustained delivery of acyclovir up to 8 hours, it is reasonable to assume based on the drug load and the continued presence of the non volatile solvent that the delivery of acyclovir would continue at the reported flux values for as long as the subject desires to leave the adhesive solidifying formulation affixed to the skin.

### Example 12

A formulation similar to Example 4 (with no acyclovir) is applied onto a human skin surface, resulting in a thin, transparent, flexible, and stretchable film. After a few minutes of evaporation of the volatile solvent (ethanol), a solidified adhesive layer that is peelable is formed. The stretchable film has good adhesion to the skin and did not separate from the skin, and could easily be peeled away from the skin. The absence of acyclovir is expected to have minimal to no impact on the physical and wear properties of the coherent solid because it is present at such low concentration, when present.

### Examples 13-14

Anti-fungal formulations are prepared and a qualitative assessment of peel flexibility and viscosity are evaluated. The formulation components are presented in Table 6 below.

TABLE 6

| Example | 13 | 14 |
|---|---|---|
| Components | Parts by Weight | |
| Eudragit RL-PO | 3.8 | 4.2 |
| Isostearic Acid | 2 | 2.2 |
| Ethanol | 5.3 | 3.8 |
| Neutrol TE Polyol | 1 | 1 |
| Econazole | 0.09 | 0.1 |

The formulation in Example 13 has a low viscosity that was lower than may be desirable for application on a nail or skin surface. The time to form a solidified peel with this formulation is longer than the desired drying time. The formulation in Example 14 had an increase in the amount of solidifying agent (Eudgragit RL-PO) and decrease in amount of ethanol, which improves the viscosity and drying time. Example 14 has a viscosity suitable for application and an improved drying time.

While the invention has been described with reference to certain preferred embodiments, those skilled in the art will appreciate that various modifications, changes, omissions, and substitutions can be made without departing from the spirit of the invention. It is therefore intended that the invention be limited only by the scope of the appended claims.

What is claimed is:

1. An adhesive solidifying formulation for treating a skin or nail infection, comprising:
a) a drug for treating a skin or nail infection;
b) a solvent vehicle, comprising:
i) a volatile solvent system including at least one volatile solvent, and
ii) a non-volatile solvent system including at least one non-volatile solvent; and
c) a solidifying agent,
wherein the formulation has a viscosity suitable for application and adhesion to a skin or nail surface prior to evaporation of the volatile solvent system, the formulation applied to the skin or nail surface forms a solidified layer after at least partial evaporation of the volatile solvent system, and the drug continues to be dermally delivered after the volatile solvent system is evaporated.

2. A formulation as in claim 1, wherein the non-volatile solvent system acts as a plasticizer for the solidifying agent.

3. A formulation as in claim 1, wherein the skin or nail infection is a viral infection, a bacterial infection or a fungal infection.

4. A formulation as in claim 1, wherein the nail infection is a fungal infection.

5. A formulation as in claim 1, wherein the non-volatile solvent system is flux-enabling for the drug.

6. A formulation as in claim 1, wherein the formulation further comprises an additional agent which is added to increase adhesion of the formulation when applied to a body surface, said additional agent including a member selected from the group consisting of copolymers of methylvinyl ether and maleic anhydride, polyethylene glycol and polyvinyl pyrrolidone, gelatin, low molecular weight polyisobutylene rubber, copolymer of acrylsan alkyl/octylacrylamido, aliphatic resins, aromatic resins, and combinations thereof.

7. A formulation as in claim 1, wherein the volatile solvent system includes a member selected from the group consisting of water, ethanol, isopropyl alcohol, and combinations thereof.

8. A formulation as in claim 1, wherein the volatile solvent system includes at least one solvent more volatile than water, and includes at least one member selected from the group consisting of ethanol, isopropyl alcohol, dimethyl ether, diethyl ether, butane, propane, isobutene, 1,1, difluoroethane, 1,1,1,2 tetrafluorethane, 1,1,1,2,3,3,3-heptafluoropropane, 1,1,1,3,3,3 hexafluoropropane, ethyl acetate, acetone, denatured alcohol, methanol, propanol, isobutene, pentane, hexane, cytopentasiloxane, cyclomethicone, methyl ethyl ketone, and combinations thereof.

9. A formulation as in claim 1, wherein the non-volatile solvent system includes at least one solvent selected from the group consisting of isostearic acid, oleic acid, olive oil, trolamine, glycerol, propylene glycol, isostearic acid, oleic acid, propylene glycol, trolamine, tromethamine, triacetin, sorbitan monolaurate, sorbitan monooleate, sorbitan monopalmitate, benzoic acid, dibutyl sebecate, diglycerides, dipropylene glycol, eugenol, fatty acids, isopropyl myristate, mineral oil, oleyl alcohol, vitamin E, triglycerides, sorbitan fatty acid surfactants, triethyl citrate, 1,2,6-hexanetriol, alkyltriols, alkyldiols, tocopherol, p-propenylanisole, anise oil, apricot oil, dimethyl isosorbide, alkyl glucoside, benzyl alcohol, bees wax, benzyl benzoate, butylene glycol, caprylic/capric triglyceride, caramel, cassia oil, castor oil, cinnamaldehyde, cinnamon oil, clove oil, coconut oil, cocoa butter, cocoglycerides, coriander oil, corn oil, corn syrup, cottonseed oil, cresol, diacetin, diacetylated monoglycerides, diethanolamine, diglycerides, ethylene glycol, eucalyptus oil, fat, fatty alcohols, flavors, liquid sugars ginger extract, glycerin, high fructose corn syrup, hydrogenated castor oil, IP palmitate, lemon oil, lime oil, limonene, monoacetin, monoglycerides, nutmeg oil, octyldodecanol, orange oil, palm oil, peanut oil, PEG vegetable oil, peppermint oil, petrolatum, phenol, pine needle oil, polypropylene glycol, sesame oil, spearmint oil, soybean oil, vegetable oil, vegetable shortening, wax, 2-(2-(octadecyloxy)ethoxy)ethanol, benzyl benzoate, butylated hydroxyanisole, candelilla wax, carnauba wax, ceteareth-20, cetyl alcohol, polyglyceryl, dipolyhydroxy stearate, PEG-7 hydrogenated castor oil, diethyl phthalate, diethyl sebacate, dimethicone, dimethyl phthalate, PEG fatty acid esters, PEG-stearate, PEG-oleate, PEG laurate, PEG fatty acid diesters, PEG-dioleate, PEG-distearate, PEG-castor oil, glyceryl behenate, PEG glycerol fatty acid esters, PEG glyceryl laurate, PEG glyceryl stearate, PEG glyceryl oleate, lanolin, lauric diethanolamide, lauryl lactate, lauryl sulfate, medronic acid, multisterol extract, myristyl alcohol, neutral oil, PEG-octyl phenyl ether, PEG-alkyl ethers, PEG-cetyl ether, PEG-stearyl ether, PEG-sorbitan fatty acid esters, PEG-sorbitan diisosterate, PEG-sorbitan monostearate, propylene glycol fatty acid esters, propylene glycol stearate, propylene glycol, caprylate/caprate, sodium pyrrolidone carboxylate, sorbitol, squalene, triglycerides, alkyl aryl polyether alcohols, polyoxyethylene derivatives of sorbitan-ethers, saturated polyglycolyzed C8-C10 glycerides, N-methylpyrrolidone, honey, polyoxyethylated glycerides, dimethyl sulfoxide, azone, dimethylformamide, N-methyl formamaide, fatty acid esters, fatty alcohol ethers, alkyl-amides, N-methylpyrrolidone, ethyl oleate, polyglycerized fatty acids, glycerol monooleate, glyceryl monomyristate, glycerol esters of fatty acids, silk amino acids, PPG-3 benzyl ether myristate, Di-PPG2 myreth 10-adipate, honeyquat, sodium pyroglutamic acid, abyssinica oil, dimethicone, macadamia nut oil, limnanthes alba seed oil, cetearyl alcohol, PEG-50 shea butter, shea butter, aloe vera juice, phenyl trimethicone, hydrolyzed wheat protein, and combinations thereof.

10. A formulation as in claim 1, wherein the solidifying agent includes at least one member selected from the group consisting of polyvinyl alcohol, esters of polyvinylmethylether/maleic anhydride copolymer, neutral copolymers of butyl methacrylate and methyl methacrylate, dimethylaminoethyl methacrylate-butyl methacrylate-methyl methacrylate copolymers, ethyl acrylate-methyl methacrylate-trimethylammonioethyl methacrylate chloride copolymers, prolamine, pregelatinized starch, ethyl cellulose, fish gelatin, gelatin, acrylates/octylacrylamide copolymers, ethyl cellulose, hydroxy ethyl cellulose, hydroxy methyl cellulose, hydroxy propyl cellulose, hydroxypropyl methyl cellulose, carboxymethyl cellulose, methyl cellulose, polyether amides, corn starch, pregelatinized corn starch, polyether amides, shellac, polyvinyl pyrrolidone, polyisobutylene rubber, polyvinyl acetate phthalate, ammonia methacrylate, carrageenan, cellulose acetate phthalate aqueous, carboxy polymethylene, cellulose acetate (microcrystalline), cellulose polymers, divinyl benzene styrene, ethylene vinyl acetate, silicone, guar gum, guar rosin, gluten, casein, calcium caseinate, ammonium caseinate, sodium caseinate, potassium caseinate, methyl acrylate, microcrystalline wax, polyvinyl acetate, PVP ethyl cellulose, acrylate, PEG/PVP, xantham gum, trimethyl siloxysilicate, maleic acid/anhydride copolymers, polacrilin, poloxamer, polyethylene oxide, poly glactic acid/poly-I-lactic acid, turpene resin, locust bean gum, acrylic copolymers, polyurethane dispersions, dextrin, polyvinyl alcohol-polyethylene glycol co-polymers, methacrylic acid-ethyl acrylate copolymers, methacrylic acid and methacrylate based polymers, and combinations thereof.

11. A formulation as in claim 1, wherein the solidifying agent includes a member selected from the group of methacrylic polymer, a methacrylic acid-ethyl acrylate copolymer, a polyvinyl alcohol-polyethylene glycol copolymer, a methacrylic acid copolymer, an aminoalkyl methacrylate copolymer, an ammonioalkyl methacrylate copolymer, and combinations thereof.

12. A formulation as in claim 1, wherein the drug includes multiple drugs.

13. A formulation as in claim 1, wherein the drug is an anti-viral agent.

14. A formulation as in claim 13, wherein the anti-viral agent includes a member selected from the group consisting of acyclovir, penciclovir, famciclovir, valacyclovir, behenyl alcohol, trifluridine, idoxuridine, cidofovir, gancyclovir, podofilox, podophyllotoxin, abacavir, delavirdine, didanosine, efavirenz, lamivudine, nevirapine, stavudine, zalcitabine, zidovudine, amprenavir, indinavir, nelfinavir, ritonavir, saquinavir, amantadine, interferon, oseltamivir, ribavirin, rimantadine, zanamivir, and combinations thereof.

15. A formulation as in claim 1, wherein the drug is for treating herpes infection, a cold sore, genital herpes infection, a nail fungal infection, or a skin fungal infection.

16. A formulation as in claim 1, wherein the drug is an antifungal agent.

17. A formulation as in claim 16, wherein the antifungal agent includes a member selected from the group consisting of amorolfine, butenafine, naftifine, terbinafine, fluconazole, itraconazole, ketoconazole, posaconazole, ravuconazole, voriconazole, clotrimazole, butoconazole-, econazole, miconazole, oxiconazole, sulconazole, terconazole, tioconazole, caspofungin, micafungin, anidulafingin, amphotericin B, nystatin, pimaricin, griseofulvin, ciclopirox olamine, haloprogin, tolnaftate, and undecylenate, and combinations thereof.

18. A formulation as in claim 17, wherein the antifungal agent is terbinafine.

19. A formulation as in claim 1, wherein the formulation further includes a protectant comprising a member selected from the group consisting of allantoin, calamine, cod liver oil, dimethicone, kaolin, lanolin, mineral oil, petrolatum, talc, topical starch, white petrolatum, zinc oxide, and combinations thereof.

20. A formulation as in claim 1, wherein the drug is an antibacterial agent.

21. A formulation as in claim 20, wherein the antibacterial agent includes a member selected from the group consisting of erythromycin, clindamycin, tetracycline, bacitracin, neomycin, mupirocin, polymyxin B, quinolones such as ciproflaxin, and combinations thereof.

22. A formulation as in claim 1, wherein the drug is an immune modulating agent such as imiquimod.

23. A formulation as in claim 1, wherein the solidified layer is sufficiently flexible and adhesive to the skin or nail such that when applied to the skin or nail at a stretchable surface, the solidified layer will remain intact on the skin or nail upon stretching of the skin or nail.

24. A formulation as in claim 1, wherein the formulation is formulated to deliver the drug at a therapeutically effective rate for at least 2 hours following the formation of the solidified layer, or at least 4 hours following the formation of the solidified layer, or at least 8 hours following the formation of the solidified layer, or at least 12 hours following the formation of the solidified layer, or at least 24 hours following the formation of the solidified layer.

25. A formulation as in claim 1, wherein the non-volatile solvent system is capable of causing human skin irritation and at least one non-volatile solvent of the non-volatile solvent system is capable of reducing the skin irritation.

26. A formulation as in claim 1, wherein the solidified layer is formed within about 15 minutes of the application to the skin or nail surface under standard skin and ambient conditions.

27. A formulation as in claim 1, wherein the formulation has an initial viscosity prior to skin or nail application from about 100 to about 3,000,000 centipoises, or from about 1,000 to about 1,000,000 centipoises.

28. A formulation as in claim 1, wherein the volatile solvent system is from about 10 wt % to about 85 wt % of the formulation, or from about 20 wt % to about 50 wt % of the formulation.

29. A formulation as in claim 1, wherein the non-volatile solvent system includes multiple non-volatile solvents and at least one of the non-volatile solvents improves the compatibility of the non-volatile solvent system with the solidifying agent.

30. A formulation as in claim 1, wherein the formulation applied to the skin or nail surface forms a solidified layer that is coherent, flexible, and continuous after at least partial evaporation of the volatile solvent system.

31. A formulation as in claim 1, wherein the solidified layer, upon formation, is a soft, coherent solid that is peelable from a skin or nail surface as a single piece or as only a few large pieces relative to the application size.

32. A formulation as in claim 1, wherein the weight ratio of the non-volatile solvent system to the solidifying agent is from about 0.5:1 to about 2:1.

33. A formulation as in claim 1, wherein the solidified layer can be stretched in at least one direction by 5% without cracking, breaking and/or separating from a skin or nail surface.

34. A method of treating a skin or nail infection, comprising:
a) applying an adhesive solidifying formulation as a layer having a thickness from about 0.05 mm to about 1 mm to a skin or nail surface over an infected site, the adhesive formulation comprising:
i) a drug for treating an infection;
ii) a solvent vehicle, comprising:
a volatile solvent system including at least one volatile solvent, and
a non-volatile solvent system including at least one non-volatile solvent, wherein the non-volatile solvent system is capable of facilitating the delivery of the drug at therapeutically effective rates over a sustained period of time; and
iii) a solidifying agent,
wherein the formulation has a viscosity suitable for application and adhesion to the skin or nail site prior to evaporation of the volatile solvent system, and the drug continues to be dermally delivered after the volatile solvent system is evaporated;
b) solidifying the formulation to form a soft, coherent, solidified, layer on the skin or nail surface by at least partial evaporation of the volatile solvent system; and
c) dermally delivering the drug from the solidified layer to the infected site at therapeutically effective rates over a sustained period of time.

35. A soft, coherent solidified layer for treating a skin or nail infection, comprising:
a) a drug that is effective for treating a skin or nail infection;
b) a non-volatile solvent system including at least one non-volatile solvent, wherein the non-volatile solvent system facilitates the delivery of the drug at a therapeutically effective rate over a sustained period of time; and
c) a solidifying agent.
